(19) United States
(12) Patent Application Publication
(10) Pub. No.: US 2014/0076335 A1
(43) Pub. Date: Mar. 20, 2014

(54) MOUTH SEAL DEVICE FOR EMPLOYMENT WITH POSITIVE AIRWAY PRESSURE THERAPY

(71) Applicant: Todd D. Morgan, Carlsbad, CA (US)

(72) Inventor: Todd D. Morgan, Carlsbad, CA (US)

(21) Appl. No.: 13/623,481

(22) Filed: Sep. 20, 2012

(51) Int. Cl. A61C 5/14; A61M 16/00

(52) U.S. Cl. 128/860

(57) ABSTRACT

A device for significantly reducing the leakage of air through the mouth of a patient undergoing positive airway pressure (PAP) therapy. The device provides a mouth seal which is fitted to the teeth and gums of the user and a tongue receiving portion which positions the tongue in a forward position in a manner to significantly reduce or prevent the passage of air out of the mouth.

This application claims priority to U.S. Provisional Patent Application Ser. No. 61/537,003 filed on Sep. 20, 2011 which is incorporated herein in it's entirety by reference.

### BACKGROUND OF THE INVENTION

1. Field of the Invention

This invention relates generally to an apparatus for optimizing the effectiveness of positive airway pressure devices used in the treatment of obstructive sleep apnea in human beings, and more particularly to a structure which reduces air leakage through the mouth, thereby permitting reduction of the required air pressure applied to the user.

2. Prior Art

Sleep apnea occurs when a person momentarily stops breathing or has diminished breathing during sleep. The severity of sleep apnea can vary depending on the frequency that breathing actually stops or slows. In general, the pause in breathing may last from seconds to minutes and can occur up to 30 times or more an hour.

One common type of sleep apnea, called obstructive sleep apnea (OSA), is the result of blocked airflow during sleep. Such blockages can result from narrowing in the pharyngeal airway arising from natural muscle relaxation during sleep. Anatomical factors such as obesity can also contribute to OSA due to the low muscle tone around the airway. This reduces oxygen in the blood and causes loud snoring and arousal from sleep. In some cases, severe sleep apnea can present serious risk of injury arising from interrupted oxygen supply to the body.

Conventional treatments of sleep apnea involve lifestyle changes such as avoiding alcohol or muscle relaxants, losing weight, and quitting smoking. It is also known that maintaining the lower jaw forward and allowing the tongue to maintain a forward position will further reduce the obstruction of the pharyngeal airway. However, for those unable to pursue the conventional methods of treatment, medical therapies have been developed to protect patients suffering from sleep apnea from the interrupted breathing process.

One such therapy is positive airway pressure (PAP), or continuous positive airway pressure (CPAP) therapy. PAP therapy uses a device to deliver a stream of compressed air to the nose of a patient afflicted with OSA to reduce any collapse of the pharyngeal airway when the patient inhales. In effect, this pressurized air provides a pneumatic splint to the airway, thereby keeping it open for unobstructed breathing.

A common PAP device features a mask which covers the nose and is referred to as a nasal CPAP or NCPAP. NCPAP is an effective non-surgical treatment for OSA and has found broad application. For some patients, improvement in the quality of sleep and quality of life resulting from NCPAP therapy can be noticed after a single night.

However, one of the problems encountered in the use of existing NCPAP devices is the escape of air through the oral cavity of the patient, reducing the pressure in the user's airways and, consequently allowing the muscles and soft tissue to relax and again obstruct the airway and greatly reducing the effectiveness of the device. Maintaining an effective pressure throughout the night while the patient is asleep is the key to relief of OSA, and the lose of air pressure through the mouth is a predominant problem. The need to seal the oral cavity and reduce blockage of the airway is more then evident.

Prior art has shown many attempts in the art of oral devices. U.S. Pat. No. 5,752,822 to Robson teaches a dental orthotic for positioning the tongue in a forward position. The device rests in the users mouth and provides a means for elevating the tongue from its natural resting position to allow the tongue to move forward and therefor reduce blockage of pharyngeal airway. The device to Robson however, does not provide a means for sealing the oral cavity of the user as may be desired with the employment of NCPAP machines.

U.S. Pat. No. 5,467,783 to Meade teaches a dental orthotic device for preventing backward movement of the lower jaw. However the device does not provide a means for maintaining the tongue in a substantially forward position nor provide a substantial seal of the oral cavity.

These and similar devices fall short in providing adequate reduction in airway obstruction by sealing the oral cavity, maintaining the lower jaw in a substantially forward position, and allowing the tongue to passively maintain a forward position.

As such, there is a continuing and unmet need for a dental orthotic device that can maintain the lower jaw and tongue at a substantially forward position while concurrently sealing the oral cavity. Such a device should additionally accommodate the lateral width of the tongue without constricting the tongue or restricting the natural resting position the tongue when the device is in the as used position.

The forgoing examples of related art and limitation related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

The device herein disclosed and described provides a solution to the shortcomings in prior art and achieves the above noted goals through the provision of an intra-oral appliance as a means for preventing or significantly reducing the escape of air through the user's mouth provided during NCPAP therapy.

Use of the device by the user is passive so that it continues to function effectively even while the user is asleep. By reducing air leakage via the mouth, the amount of positive pressure required to maintain an effective pneumatic splint may also be reduced.

As a secondary benefit resulting the provided reduction in applied air pressure, employment of the device herein also reduces the amount of air leaking around the seal of the mask, further enhancing the effectiveness and stability of the PAP therapy process. Further utility is found in that the PAP machine can function effectively at a lower applied air pressure, thereby being quieter and using less energy.

In a particularly preferred mode, the device includes an oral appliance which is fitted to the teeth of a patient in such a manner as to urge the jaw into a slightly forward or protruding position. The oral device is provided with an integral, central tongue receiving portion which receives the tongue of the patient in a substantially forward position. The oral appliance is closely fitted to the anatomy of the teeth, gums, and tongue of the user to provide a means for sealing the oral cavity when it is in place.

The positioning of the tongue in the tongue receiving portion in the forward position provides a means for achieving an unimpeded airflow through the pharyngeal airway with no active participation required of the patient. Once the seal is obtained, the patient can simply relax and fall asleep while the PAP process continues throughout the period of sleep.

The mouth seal device generally includes an upper U-shaped cavity portion for engaging the maxillary teeth, and a lower U-shaped cavity portion for engaging the mandibular teeth. The upper and lower portions are preferably disposed at a predetermined angle as a means for holding the user's mouth slightly open and to provide a tongue passage between the maxillary and mandibular incisors. In a first preferred mode the tongue passage includes a cavity portion having a first open end extending from a location inside the users mouth adjacent the incisors to a closed terminating end wall located substantially exterior the users mouth. The provision of the passage provides great advantage over prior art in that the tongue can be passively maintained at a substantially forward position insomuch as being outside the mouth of the user. Maintaining the tongue in this position is additionally enhanced when a positive air pressure is applied such as during NCPAP therapy.

In another preferred mode however the tongue receiving portion includes a substantially central planar member providing a means for cradling the tongue. In this mode the planar member provides a means for maintaining the tongue at a forward position while concurrently sealing the oral cavity. However this mode may or may not provide a means for maintaining the tongue in a position substantially exterior the users mouth.

The device may be constructed from a variety of materials commonly used in dentistry. In a particularly preferred method of construction of the invention, the device is customized for an individual user by a qualified dental professional by molding impressions of the teeth and recording the bite position in a conventional manner. A dental laboratory then fabricates the device based on the data provided by the dental professional using a soft acrylic or silicone material which provides a comfortable feel to the patient and which may be readily cleaned or sterilized between usages.

In yet another particularly preferred method, pre-fabricated impression trays can be provided in a variety of sizes and shapes. A qualified dental professional selects the one which best fits the user's mouth anatomy. This is also a conventional impression procedure where the dental professional prepares a customized version of the mouth seal using this tray to mold the impressions of the teeth and actual tongue shape of the patient. The height of the side walls of the tray is selected to ensure the mouth seal produced using the form has sufficient coverage of the teeth and gums to prevent air flow around its periphery.

A thermally responsive material is placed in the tray and the entire tray with liner is submerged in hot water so the liner softens. When placed in the mouth of the patient and the patient bites down, the liner then directly forms to and around the patient's teeth and gums. This process is commonly referred to as the ‘boil-and-bite’ process. When cooled, the liner material is set, retaining an impression of the patient's teeth and mouth and allows for subsequent routine placement into the mouth

In an additionally preferred method employing the boil-and-bite process, the tongue receiving cavity of the device additionally includes this aforementioned thermally responsive material. In this manner the user may additionally form the tongue receiving cavity to a specified depth and geometry. Added utility and advantage over prior art is found in that the boil-and-bite process is conventionally employed only with the teeth receiving portions of such dental orthotics and not so with tongue related portions. A user is essentially provided with unlimited ability to customize the fit and feel of the device within their mouth.

In another method for forming the invention, the mouth seal is fabricated directly in the mouth of the patient using a two part putty that hardens in a short period of time upon mixing of the two parts. A quantity of the putty is prepared and the hardening process is initiated by mixing the two parts together. That mixture is then placed into the impression tray, again in a conventional procedure. The entire form with putty is placed into the mouth of the patient, the teeth and tongue positioned appropriately, with the tongue forward to form the cradle, and the assembly allowed to harden. Once the putty has become firm, the entire assembly is removed from the patient's mount and excess material making up the form is removed by processes such as cutting, grinding, or sanding.

In application with a user undergoing PAP therapy, an oral formed according to one of the methods described above is engaged into the mouth of the user, typically directly by the user and the PAP process is started. The pressurized air provided by the PAP equipment will provide a means for enhancing the seal of the device in the users mouth and will additionally aid in maintain the tongue in the forward position in the tongue receiving portion of the device.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

It is an object of the invention to provide a mouth seal device for concurrent employment in combination with a PAP therapy device as a means for sealing the oral cavity of a user while concurrently providing a means for maintaining the tongue and lower jaw at a substantially forward position.

It is another object of the invention to provide a mouth seal device sealing the oral cavity of a user while concurrently providing a means for maintaining the lower jaw at a substantially forward position and passively maintaining the tongue at a forward position substantially exterior the users mouth.

It is yet another object of the invention to provide a mouth seal device that is custom formed to the users teeth and tongue via the conventional boil-and-bite process described above.

These and other objects, features, and advantages of the invention will be brought out in the following part of the specification, wherein detailed description is for the purpose of fully disclosing the invention without placing limitations thereon.

### BRIEF DESCRIPTION OF DRAWING FIGURES

The accompanying drawings, which are incorporated herein and form a part of the specification, illustrate some, but not the only or exclusive, examples of embodiments and/or features. It is intended that the embodiments and figures disclosed herein are to be considered illustrative rather than limiting. In the drawings:

FIG. 1 is a side, partial cross-sectional view of a patient undergoing PAP therapy according to the prior art.

FIG. 2 is a similar side, partial cross-sectional view of a patient undergoing PAP therapy with the presence of a mouth seal device in accordance with a preferred mode of the present invention.

FIG. 3 is an elevated perspective view of a preferred mode of the device formed from a relatively soft material such as acrylic or silicone.

FIG. 4 is a side view of the mouth seal device of FIG. 3.

FIG. 5 is a top view of the mouth seal device of FIG. 3.

FIG. 6 is a bottom view of the mouth seal device of FIG. 3.

FIG. 7 is rear view of the mouth seal device of FIG. 3.

FIG. 8 is elevated perspective view of another particularly preferred mode of the device employing lateral tongue receiving cavities.

FIG. 9 is a rear view of the mode of the device of FIG. 8.

FIG. 10 is a perceptive view of still yet another simplified mode of the device.

FIG. 11 is a top view of the device of FIG. 10.

FIG. 12 is a rear view of the device of FIG. 10.

FIG. 13 is a top view of the as-used mode of the device of FIG. 10.

### DETAILED DESCRIPTION OF THE PREFERRED

### Embodiments of the Invention

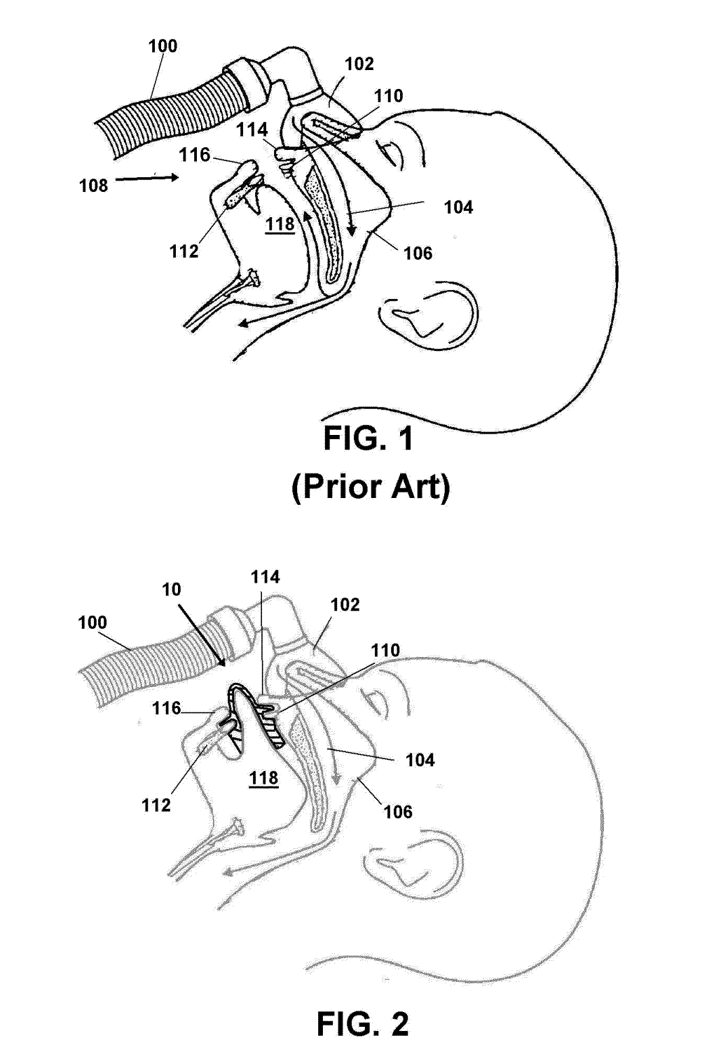

Now referring to drawings in FIGS. 1-13, wherein similar components are identified by like reference numerals, there is seen in FIG. 1 a user undergoing conventional PAP therapy employing an NCPAP device 100. The NCPAP mask 102 covers the users nose and applies pressurized air 104. The air 104 flows into the nasal respiratory airway and pharyngeal airway 106 and, as a downfall of the device 100 mentioned previously, a portion of the applied air 104 flows out through the users oral cavity and mouth 108. The mouth 108 includes the upper maxillary teeth 110, lower mandibular teeth 112, upper lip 114, lower lip 116, and tongue 118.

It is one of the objects of the present invention to provide a means for sealing the oral cavity of a user undergoing PAP therapy. Shown in FIG. 2 there is depicted the same user undergoing PAP therapy employing a particularly preferred mode of the device 10 of the present invention. Generally, the device 10 provides a seal of the users mouth, urges the lower teeth 112 and jaw substantially forward, and maintains the tongue 118 at a substantially forward position. This, as noted previously, provides a means for sealing the oral cavity, and a means for clearing any obstruction of the pharyngeal airway with no active participation required of the user. Detailed explanation of the preferred mode of the device 10 is provided shortly below.

The oral appliance device 10 in accordance with a particularly preferred mode of this invention may be constructed from a variety of materials commonly used in dentistry. In one particularly preferred method of construction of the invention, the device 10 is customized for an individual user by a qualified dental practitioner by molding impressions of the teeth and tongue and recording the bite position and tongue geometry as is conventionally known in the art. The device 10 is the fabricated based on the data provided by the practitioner using a soft acrylic or silicone material which provides a comfortable feel to the patient and which may be readily cleaned or sterilized between usages.

A more detailed description this and other preferred methods of construction of the device 10 will be set forth later in this disclosure. Briefly, other preferred methods of construction include the conventional boil-and-bite technique wherein a thermally responsive material such as ethylene-vinyl acetate, or other material known in the art, such as the commercially available ThermAcryl®, is employed to allow the user to mold impressions themselves.

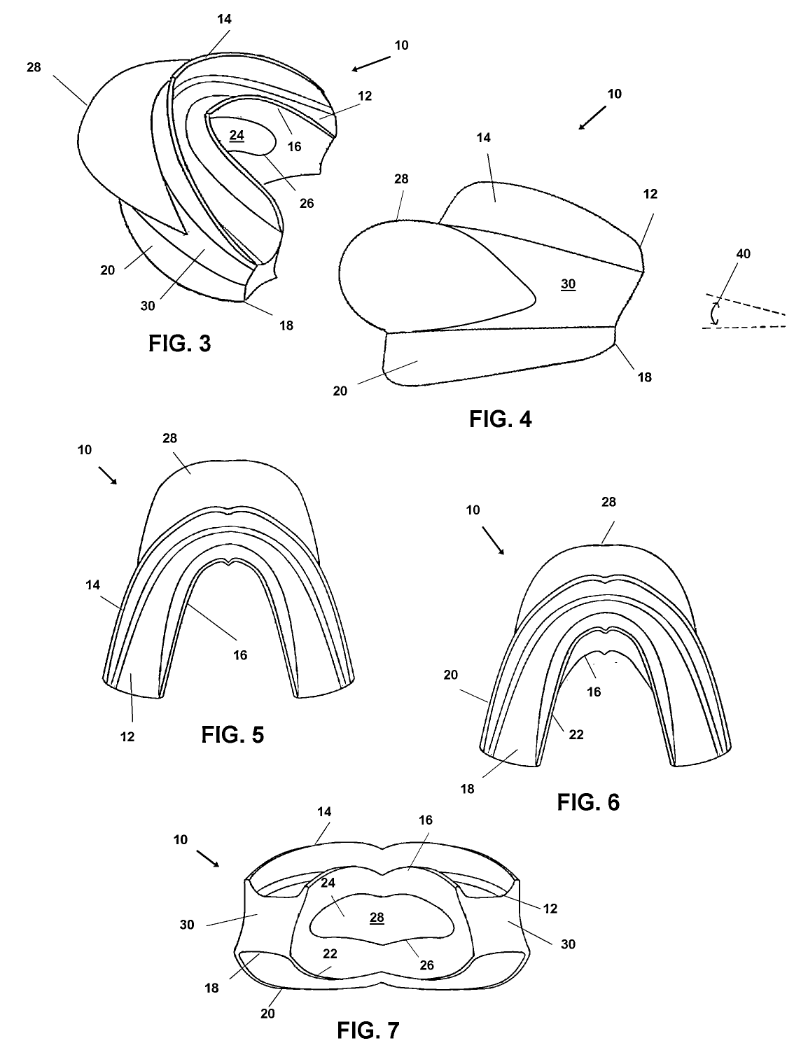

FIG. 3 shows an elevated perspective view of a particularly preferred mode of the mouth seal device 10. The device 10 is intended to be received comfortably into the mouth of a user and as such it must be noted that some modifications and alterations of the device 10 depicted may be needed to conform to the varying geometry of mouths from user to user. As such those skilled in the art will appreciate that the forgoing disclosure is provided to merely convey the overall scope and intent of the device 10 where such modifications are anticipated.

The device 10 includes and upper cavity 12 and lower cavity 18. The cavities 12, 18 are generally U-shaped and are intended to engage and conform to the maxillary 110 and mandibular 112 teeth of the respective dental arches of a user in the as used position. The upper cavity 12 and lower cavity 18 include forward sidewalls 14, 20 and rearward sidewalls 16, 22 respectively extending therefrom which provide a means for sealing the periphery of the mouth of the user, and is discussed in more detail below.

As a further note the terms ‘forward’ and ‘rearward’, ‘front’, ‘rear’, ‘right’ and ‘left’ refer herein to the anatomical directions. The use of these terms with various components should therefor be easily understood by a person skilled in the art as related to orientation, direction, and disposition.

As can be seen clearly in the side view of FIG. 4 there is an additional sidewall portion 30 communicating between the upper cavity 12 and lower cavity 18. The sidewall 30 communicates between the upper and lower cavities 12, 18 respectively forming the unitary structure of the device 10 as shown. It must be noted that the opposite side view is merely a mirror image of the current figure. The upper 12 and lower 18 cavities are configured on the device 10 to maintain the users mouth 108 in a slightly open position insofar as to allow the user to extend the tongue 118 forward between the upper maxillary teeth 110 and lower mandibular teeth 112. To provide a comfortable open position the upper cavity 12 is preferably formed at an angle 40 relative the lower cavity 18. Allowing the users to maintain a slightly open mouth position further aids in maintaining the tongue 118 in a forward position for sealing the oral cavity and opening the pharyngeal airway. This angle 40 is preferably between 0 and 30 degrees, however, other values outside this range may be employed and are anticipated.

There is included a tongue receiving cavity 24 communicating axially between the upper cavity 12 and lower cavity 18 within the body of the device, as can be seen more clearly in the rear view of FIG. 7. The cavity 24 generally includes an open end 26 adapted to receive the users tongue for insertion into the cavity 24. The cavity 24 extends forward to a substantially curved terminating endwall 28.

In the as worn position of the device 10 in the current disclosed preferred mode, the endwall 28 will be disposed substantially forward the U-shaped cavities 12, 18 such as to allow the tip of the tongue to be passively positioned up to the endwall 28 and therefor outside of the mouth 108, when the user is asleep and employing NCPAP therapy. The endwall 28 generally conforms to the curvature and contour of the human tongue as to comfortably receive the tongue in a rested position.

However, in other modes of the device 10, it may be preferred that the endwall 28 is substantially inline with the forward walls 14, 20 of the upper 12 and lower 18 cavities respectively, as opposed to substantially forward the cavities 12, 18 as shown. This would allow the tongue to still maintain a substantially forward position from its relaxed position with the tip of the tongue behind the lower dental arch but not outside the mouth 108.

As such it must be noted that the forward position of the endwall 28 may be further or closer to the forward walls 14, 20 depending on a particular users needs and should therefor not be considered limiting by the depictions. However again noting it is particularly preferred that the endwall 28 extends substantially forward of the cavity portions 12, 18 to allow the user to maintain the tongue 118 in a forward position outside of the users mouth 108. Further, as will be set forth later in this disclosure, the endwall 28 of the cavity 24 can be custom fitted to a users tongue geometry for improved fit and comfort.

Shown in the top and bottom views of FIG. 5 and FIG. 6, the lower cavity 18 is positioned substantially forward of the upper cavity 12 as to urge the jaw and lower mandibular teeth 112 in a substantially forward position providing a means for opening the pharyngeal airway 106. This additionally provides a means for aiding in maintaining the tongue in a forward position.

In the as used position of the device 10 shown previously in FIG. 2, the forward wall 14 of the upper cavity 12 is engaged between the upper lip 114 and maxillary teeth 110 and gums, while the forward wall 20 of the lower cavity 12 is engaged between the lower lip 116 and mandibular teeth 112 and gums, providing a means for sealing the periphery of the oral cavity.

This, along with the achieved forward position of the tongue 118 in the receiving cavity 24 significantly reduces or at least partially blocks the flow of air around the periphery of the seal provided by the forward walls 14, 20, wherein such a seal is most preferred with concurrent employment of NCPAP therapy.

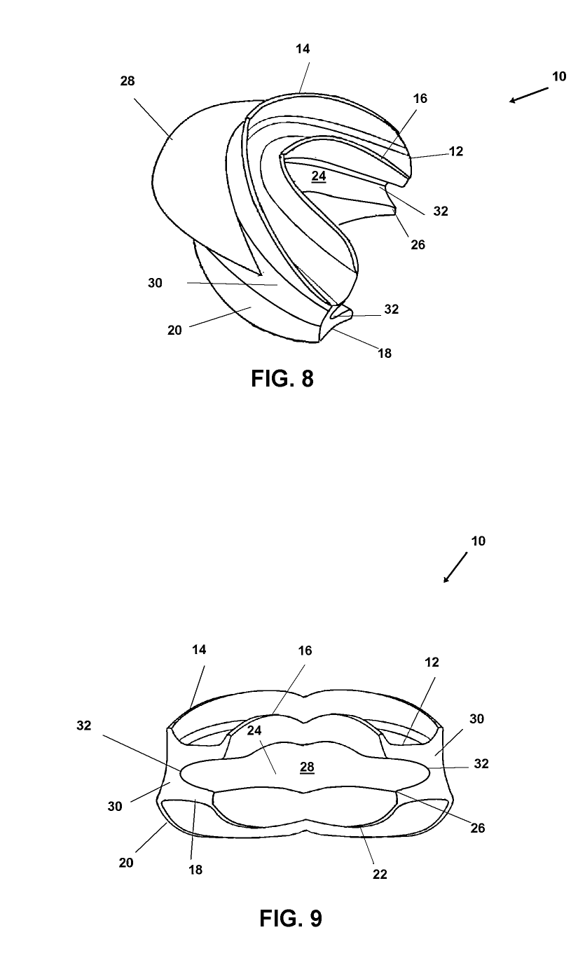

FIG. 8 shows an elevated perspective view of another particularly preferred mode of the device 10. In this mode, the device 10 further includes lateral cavity portions 32 communicating with the tongue receiving cavity 24. The lateral cavity portions 32 provides a means for comfortably receiving the users tongue 118 by accommodating the lateral width of the tongue within the cavities 32. Shown clearly in the rear view in FIG. 9, the lateral cavities 32 can be seen extending into the sidewalls 30. Such cavities 32 provide added utility and are advantageous over prior art in that the natural geometry of the tongue in a rested position can be comfortably received into the now larger cavity 24 including the lateral portions 32.

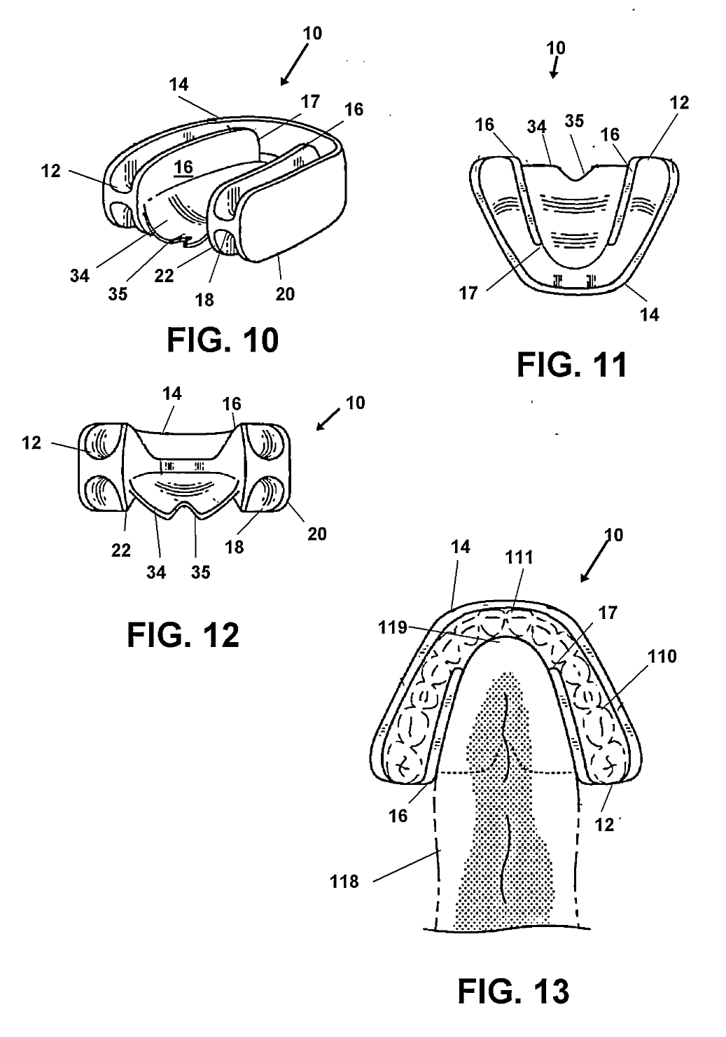

FIGS. 10-13 show still yet another preferred mode of the mouth seal device 10. This mode additionally provides an upper cavity 12 and a lower cavity 18 for engaging the maxillary 110 and mandibular 112 teeth respectively. There is further shown forward walls 14, 20 and rearward walls 16, 22 engaged to and extending from the respective upper and lower cavities 12, 18. However, in this mode there is alternatively a tongue receiving cradle defined by a partition 34 communicating in the substantially central space between the left and right sides of rearward walls 16, 22, as one could clearly see from the figure. The cradle partition 34 additionally includes a notch 36 located near the rear of the device 10 for comfortably receiving the frenulum of the tongue 118 when the tongue 118 is in a rested position on the cradle 34 as shown in the top view of FIG. 13.

In use, the current mode of the device 10 allows the user to position their tongue 118 at a substantially forward position such as to allows the tip 119 of the tongue 118 to engage the rear side of the incisors 111. There is shown a substantial gap 17 located between the left and right sides of the rearward wall 16 of the upper cavity 12 that allows the tongue to extend to such a forward position.

The disclosed preferred modes of the present invention have been shown to significantly seal the oral cavity of a user as is desired with employment of an NCPAP device. Those skilled in the art will appreciate various methods for constructing or forming the device 10, however below are provided a few particularly preferred methods of forming such a mouth seal device 10 in accordance with the above noted preferred modes.

As previously stated, in one particularly preferred method of construction of the present invention, the device 10 is customized for an individual user by a dental practitioner by conventional means of molding impressions of the teeth and recording the bite position. The device 10 may then be fabricated based on the data provided using a soft acrylic or silicone material, or other suitable material known in the art which provides a comfortable fit and feel to the patient and which may be readily cleaned or sterilized between usages.

As an alternative to the method described above, an additional preferred method of forming the device includes providing pre-fabricated mouth seal impression trays in a variety of sizes and shapes. In this current method a practitioner selects the one which best fits the user's mouth anatomy. This is also a conventional impression procedure where the dentist prepares a customized version of the mouth seal device 10 using this tray to mold the impressions of the teeth and actual tongue shape of the patient. The height of the side walls of the tray is selected to ensure the mouth seal produced using the form has sufficient coverage of the teeth and gums to prevent air flow around its periphery of the oral cavity.

A liner employing thermally responsive material is placed in the tray and the entire tray with liner is submerged in hot water so the liner softens. Such thermally responsive material can be any thermally responsible material known in the art. When placed in the mouth of the user and the user bites down, the liner then directly forms to and around the user's teeth and gums. This is commonly referred to as the ‘boil-and-bite’ process. When cooled, the liner material is set, retaining an impression of the user's teeth and mouth and allows for subsequent routine placement into the mouth.

In yet another method for forming the preferred modes of the invention, the mouth seal device 10 is fabricated directly in the mouth of the patient using a two part putty that hardens in a short period of time upon mixing of the two parts. A quantity of the putty is prepared and the hardening process is initiated by mixing the two parts together. That mixture is then placed into the impression tray, again in a conventional procedure. The entire form with putty is placed into the mouth of the user, the teeth and tongue positioned appropriately, with the tongue forward to form the cradle, and the assembly allowed to harden. Once the putty has become firm, the entire assembly is removed from the patient's mount and excess material making up the form is removed by processes such as cutting, grinding, or sanding.

In still yet another preferred method, the user selects from a plurality of different shape and sizes of pre-fabricated devices formed either partially or in entirety of thermally responsive material. In this method, the user can proceed with the conventional boil-and-bite technique for retaining an impression of the teeth and mouth. Additionally, for the preferred modes described in FIGS. 3-9, the endwall 28 may additionally be formed of this thermally responsive material wherein the user will simply press the tongue on the endwall 28 during the boil-and-bite process providing a means for forming the cavity 24 to the desired depth.

For the current method of construction and for all mode of the device 10, the device 10 in its entirety may be formed of such thermally responsive material as to allow the user to employ the boil-and-bite technique for forming all aspects of the invention including the angle 40. Alternatively, the device 10 may include a predetermined endwall 28 distance yet provide thermally responsive material for the cavities 12, 18 for making teeth impressions but not a tongue impression.

Further, it must be noted that the above outlined methods may be used in conjunction with each other wherein some components of the device 10 are formed employ a technique from one of the above methods while other components of the device 10 are formed employing another. For example, the cavities 12, 18 may be formed via the conventional molding impressions done by a dental practitioner while the endwall 28 and cavity 24 may be formed via the boil-and-bite technique employing thermally responsive material.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed:

1. An oral appliance configured for an engagement within the oral cavity defining the mouth of a user in an as-worn position, comprising;
a body,
said body having a substantially U-shaped upper cavity positioned on said body and configured to receive maxillary teeth of a user;
said body having a substantially U-shaped lower cavity positioned on said body, opposite said upper cavity, said lower cavity configured to receive mandibular teeth of said user;
said lower cavity positioned on said body substantially forward of said upper cavity with said appliance positioned in said as-worn position in the mouth of a user;
a forward wall positioned on said body;
means for passively maintaining the tongue of said user in a substantially forward position with a the tip of said tongue positioned toward from said mandibular teeth of said user while said appliance is in said as-worn position; and
said oral appliance engaged in said as-worn position with said maxillary teeth engaged with said upper cavity, positioning and said mandibular teeth engaged with said lower cavity, positioning said forward wall to a sealing position in-between gums of said maxillary teeth and the interior of the upper lip and in-between gums of said mandibular teeth and the interior of the lower lip of said mouth, said forward wall in said sealing position providing means for sealing the oral cavity of said user;
said forward position of said lower cavity with said appliance in said as worn position providing means for maintaining the lower jaw of said user substantially forward from its normal position; and
said tongue in said substantially forward position thereby providing a means for translating a rear portion of said tongue opposite said tip and maintaining an unobstructed passage for air to communicate through said pharyngeal airway of said user.

2. The oral appliance of claim 1 wherein said means for passively maintaining the tongue of said user in a substantially forward position comprises:
a tongue receiving cavity defined by a sidewall and axially disposed between said upper cavity and said lower cavity, said tongue receiving cavity having an open end and a endwall portion of said sidewall opposite said open end;
said endwall positioned at a front location distanced further from said open end and said forward wall; and
whereby said tongue is insertable in a communication through said open end to an inserted position in said tongue receiving cavity with a tip of said tongue adjacent to said front location.

3. The oral appliance of claim 2 further comprising;
laterally extending cavity portions communicating with said tongue receiving cavity; and
whereby said laterally extending cavity portions provide a means for accommodating the width of said tongue.

4. The oral appliance of claim 2 further comprising;
said front location of being exterior of said users mouth when in said appliance is engaged in said as worn position.

5. The oral appliance of claim 3 further comprising;
said front location of being exterior of said users mouth when in said appliance is engaged in said as worn position.

6. The oral appliance of claim 1 further comprising:
said upper cavity and said lower cavity formed from a thermally responsive material which softens when heated; and
whereby said user can form cooperative impressions of said maxillary and said mandibular teeth in said upper and lower cavities by imparting a forcing thereof into said thermally responsive material.

7. The oral appliance of claim 2 further comprising:
said upper cavity and said lower cavity formed from a thermally responsive material which softens when heated; and
whereby said user can form cooperative impressions of said maxillary and said mandibular teeth in said upper and lower cavities by imparting a forcing thereof into said thermally responsive material.

8. The oral appliance of claim 3 further comprising:
said upper cavity and said lower cavity formed from a thermally responsive material which softens when heated; and
whereby said user can form cooperative impressions of said maxillary and said mandibular teeth in said upper and lower cavities by imparting a forcing thereof into said thermally responsive material.

9. The oral appliance of claim 4 further comprising:
said upper cavity and said lower cavity formed from a thermally responsive material which softens when heated; and
whereby said user can form cooperative impressions of said maxillary and said mandibular teeth in said upper and lower cavities by imparting a forcing thereof into said thermally responsive material.

10. The oral appliance of claim 5 further comprising:
said upper cavity and said lower cavity formed from a thermally responsive material which softens when heated; and
whereby said user can form cooperative impressions of said maxillary and said mandibular teeth in said upper and lower cavities by imparting a forcing thereof into said thermally responsive material.

11. The oral appliance of claim 2 further comprising:
said endwall formed from a thermally responsive material; and
whereby said user can form said endwall to any desired said forward position by a forcing of said tip of said tongue against said endwall while in a heated state.

12. The oral appliance of claim 4 further comprising;
said endwall formed from a thermally responsive material; and
whereby said user can form said endwall to any desired said forward position by a forcing of said tip of said tongue against said endwall while in a heated state.

13. The oral appliance of claim 5 further comprising;
said endwall formed from a thermally responsive material; and
whereby said user can form said endwall to any desired said forward position by a forcing of said tip of said tongue against said endwall while in a heated state.

14. The oral appliance of claim 7 further comprising;
said endwall formed from a thermally responsive material; and
whereby said user can form said endwall to any desired said forward position by a forcing of said tip of said tongue against said endwall while in a heated state.

15. The oral appliance of claim 8 further comprising;
said endwall formed from a thermally responsive material; and
whereby said user can form said endwall to any desired said forward position by a forcing of said tip of said tongue against said endwall while in a heated state.

16. The oral appliance of claim 9 further comprising;
said endwall formed from a thermally responsive material; and
whereby said user can form said endwall to any desired said forward position by a forcing of said tip of said tongue against said endwall while in a heated state.

17. The oral appliance of claim 10 further comprising;
said endwall formed from a thermally responsive material; and
whereby said user can form said endwall to any desired said forward position by a forcing of said tip of said tongue against said endwall while in a heated state.

18. The oral appliance of claim 1 further comprising;
said upper cavity disposed at an angle between 0 and 30 degrees relative said lower cavity.

19. The oral appliance of claim 4 further comprising;
said upper cavity disposed at an angle between 0 and 30 degrees relative said lower cavity.

20. The oral appliance of claim 5 further comprising;
said upper cavity disposed at an angle of between 0 and 30 degrees relative said lower cavity.
